(19) United States
(12) Patent Application Publication
(10) Pub. No.: US 2010/0114092 A1
(43) Pub. Date: May 6, 2010

(54) BIPOLAR INSTRUMENT AND METHOD FOR ELECTROSURGICAL TREATMENT OF TISSUE

(76) Inventors: Florian Eisele, Tubingen (DE); Daniel Schäller, Tubingen (DE); Matthias Voigtländer, Gomaringen (DE)

Correspondence Address: DICKSTEIN SHAPIRO LLP, 1825 EYE STREET NW, Washington, DC, 20006-5403, US

(21) Appl. No.: 12/524,433

(22) PCT Filed: Jan. 25, 2008
(86) PCT No.: PCT/EP2008/000603

§ 371 Date: Jan. 15, 2010

(30) Foreign Application Priority Data
Jan. 25, 2007 (DE) 10 2007 003 836.6

(51) Int. Cl. A61B 18/14

(52) U.S. Cl. 606/41

(57) ABSTRACT

A bipolar surgical instrument comprising an electrode means connected to a high-frequency generator for generating a high-frequency current at a distal end of the instrument with at least first and second electrodes for forming electric arcs therebetween. The surgical instrument further comprises a pipe, a tubular probe or a similar gas supply means with at least one lumen for supplying argon or a similar inert gas at least into a space between the first and second electrodes so that the arcs can be formed in a protective gas atmosphere. The first and second electrodes are arranged relative to one another in such away that the tissue can be heated in a currentless manner at least partly by heat generated by the arcs. A bipolar instrument prevents the tissue damage normally seen with monopolar surgical instruments and thus allows treatment to be carried out as simply and efficiently as possible. A method for electrosurgical treatment of tissue using the bipolar surgical instrument is also disclosed.

### BACKGROUND

The invention relates to a bipolar surgical instrument and to a method for electrosurgical treatment of tissue.

For many years, electrosurgical instruments have been used in high-frequency surgery both to coagulate and cut biological tissue. For coagulation a high-frequency current is passed through the tissue to be treated to cause protein calculation and dehydration. The high frequency current causes the tissue to contract in such a way that the vessels are closed and bleeding is staunched. Cutting processes are also possible by means of high-frequency current.

Electrosurgical processes can be carried out both monopolarly and bipolarly. In the case of monopolar technology the electrosurgical instrument has only a single current supply, so that the tissue to be treated (or a patient) is set to the other potential by, e.g., application of neutral electrodes. However, there is increasing attention being paid to bipolar surgical instruments which are embodied with two current supplies electrically insulated from one another. With a bipolar surgical instrument the path of current between the electrode parts can be calculated and does not extend far through the patient's body. This reduces danger of damage to, for example, pacemakers or other apparatuses connected to the patient during the operation.

The use of protective gas, in particular in argon plasma coagulation (APC), allows contact-free coagulating of tissue and serves to effectively staunch blood and devitalize tissue. In this type of coagulation inert working gas, for example argon, is passed through a gas supply means from an argon plasma coagulation instrument to the tissue to be treated, for argon metering and error monitoring. For this purpose, the gas supply means has an APC probe and an electrode for supplying a high-frequency current to the distal end of the probe. The electrode is arranged in or on the probe in such a way that it does not touch the tissue during the treatment. With the aid of the working gas and the high-frequency current, plasma can then be generated between a distal end of the probe and the tissue, so that current is applied to the tissue via the plasma. The argon plasma coagulation prevents excessive carbonization of the tissue as well as smoke and unpleasant odors.

Treatments by APC are traditionally carried out using monopolar instruments, where—as indicated above—the current has to cover large distances through a patient's body from the point of entry to the neutral electrode. The danger to patients is that improper use of the neutral electrode can lead to serious burns. Bipolar arrangements have milder effects, as the current flows only between the two electrode parts, but there remains a risk of damaging tissue and of subjecting the body to unnecessary current.

There are other drawbacks which are encountered in monopolar APC applications as well. These include, for example, neuromuscular stimulations in pulsed modes. Also, the surgical effectiveness of the monopolar APC instrument is dependent on the capacitive loading of the probe and can be affected, for example, by the length of the endoscope.

In principle, it is always difficult to control the introduction of current into the tissue to be treated. Primarily, undesirable coagulation or cutting results often result. In addition, the current introduced can be monitored only using complex means. Superficial, uniform coagulation zones are difficult to produce; fine meterability is problematic.

It is thus an object of the invention to provide a bipolar surgical instrument and a method for electrosurgical treatment of tissue where, during treatment, damage to tissue is avoided as much as possible and the treatment can be carried out as simply and efficiently as possible.

### BRIEF DESCRIPTION OF THE DRAWINGS

The invention is described in the following with the aid of embodiment examples which are explained in more detail with the aid of the figures.

FIG. 1 shows an embodiment of an exemplary bipolar surgical instrument with a current connection means and a gripping means, the instrument being guidable in a working channel of an endoscope and connected to a power source and a gas source;

FIG. 2 shows the exemplary bipolar surgical instrument according to the embodiment as shown in FIG. 1, a distal end of the instrument being shown in section;

FIG. 3 shows the distal end of the exemplary bipolar surgical instrument according to the FIG. 2 embodiment in section along the line III-III from FIG. 2;

FIG. 4 shows a further embodiment of an exemplary bipolar surgical instrument, a distal end of the instrument being shown in section;

FIG. 5 shows the distal end of the exemplary bipolar surgical instrument according to FIG. 4 in section along the line V-V from FIG. 4;

FIG. 6 shows a further embodiment of the exemplary bipolar surgical instrument, a distal end of the instrument being shown in section;

FIG. 7 shows the distal end of the exemplary bipolar surgical instrument according to FIG. 6 in section along the line VII-VII from FIG. 6;

FIG. 8 shows a further embodiment of the exemplary bipolar surgical instrument, a distal end of the instrument being shown in section;

FIG. 9 shows the distal end of the exemplary bipolar surgical instrument according to FIG. 8 in section along the line IX-IX from FIG. 8;

FIG. 10 is a schematic illustration of an electrode means;

FIG. 11 is a schematic illustration of an electrode means with a blow magnet;

FIG. 12 is a diagram to illustrate the depth of devitalization in tissue using the exemplary bipolar surgical instrument; and

FIG. 13 is a diagram to illustrate the depth of devitalization in tissue using instruments according to the prior art.

### SUMMARY OF THE INVENTION

The object of the invention is a bipolar surgical instrument for electrosurgical treatment of tissue, comprising the following:

an electrode means connected to a high-frequency generator for generating a high-frequency current at a distal end of the instrument with at least a first electrode and a second electrode for forming electric arcs between the first and second electrodes

a pipe, a tubular probe or a similar gas supply means with at least one lumen for supplying argon or a similar inert gas at least into a space between the first and second electrodes, so that the arcs can be formed in a protective gas atmosphere,

wherein the first and second electrode are arranged relative to one another in such a way that the tissue can be heated in a currentless manner at least partly by heat generated by the arcs.

A key feature of the bipolar surgical instrument consists in the fact that the instrument substantially prevents current from being introduced into the tissue—in particular in an advanced stage of the treatment. The instrument can be used to bring heat in a targeted manner up to the tissue to be treated, so that the required introduction of heat is carried out in a precise manner that is gentle on tissue. Accordingly, the advantages of monopolar APC arrangements and bipolar surgical instruments are combined.

In the case of mutually opposing electrodes around which the protective gas is situated, allowance may be made for the formation of an electric arc. A certain bulging of the arc is in this case due to the protective gas supplied and the flow of gas. Furthermore, in addition to flow mechanical factors (laminar, turbulent flow), atomic physical (ionisation, any collisional excitation caused by free electrodes in the E-field) and thermal factors also play a part. Thus, for example, thermal excitation of the gas ensures ignition at points which causes an arcuate formation of the arc. In any case, bulging of the arc toward the tissue to be treated facilitates the treatment, as the transfer of heat to the tissue is facilitated.

If the spacing between the end of the electrodes and the surface of the tissue, which is determined by the operator, is smaller than the design-defined spacing between the ends of the electrodes, two arcs are formed, in each case from the ends of the electrodes toward the tissue. This produces a locally highly delimited flow of current in the surface of the tissue and thus endogenous heat. In any event, as a result of the local delimitation, the effect remains very superficial. Even when the electrodes are applied to the surface of the tissue mini arcs are produced (due to the high voltage of the APC), however, owing to the crest factor, there are no effects of cutting and the coagulation therefore remains very superficial. At small applicator spacings there is a mixture of the effect of endogenous and exogenous heat in the tissue.

It has been found that the bipolar surgical instrument provides a depth of penetration (in principle of heat) of a few tenths of millimetres that is not significantly increased as a result of the further treatment course even over a relatively long time. In the case of conventional measures (introduction of current into the tissue), an unnecessarily deep depth of devitalization is reached and the tissue is often destroyed also in areas adjoining the treatment region.

In one embodiment the electrodes are embodied and arranged at the distal end of the instrument in such a way that they are set apart from one another by the at least one lumen and/or at least one insulation layer, at least distal ends of the electrodes each forming an active region in such a way that the arcs can be formed between the first electrode and the second electrode.

As at least the gas supply means (pipe or probe) are made generally of plastics material, ceramic or a similar insulating material (possible for all embodiments), the at least one insulation layer can be formed by the instrument or the pipe or the probe. The electrodes can then therefore be embedded, for example, into the gas supply means or into the pipe or the probe.

As the electrodes are intended to form the arcs only in a specific region, namely the active region, they can be insulated, in particular electrically, from one another. The mere arrangement of the electrodes in the lumen allows spacing, so that the formation of arcs can be avoided. Nevertheless, the formation of the arcs depends on the size of the spacing and the voltage applied. In order to avoid the formation of arcs at undesirable locations, an insulation layer can be arranged between the electrodes in such a way that only the active regions are available to form the arcs. In order to form the active regions, the electrodes protrude, for example, from the insulation layer, so that arcs can be formed between the active regions at a suitable voltage.

Generally, the electrodes are arranged in the lumen opposing one another in the direction of extension of the instrument (i.e. in the axial direction), said electrodes being set apart from one another by the lumen of the gas supply means and at least one insulation layer. The gas supply means is made generally of a plastics material, or, if appropriate, of ceramic, so that the electrodes can be arranged in this insulation pipe or tube. In this exemplary embodiment two electrodes can for example be fastened to the inner lateral surface of the pipe or tube in such a way that they diametrically oppose one another. The electrodes can for example be fastened to the inner lateral surface by an adhesive layer as the further insulation layer, wherein the adhesive is to be applied in such a way that no arcs are produced between the electrodes outside the active regions. The electrodes can also be introduced into recesses provided for this purpose, for example in the sleeve of the pipe, the active regions then protruding from the pipe in such a way that arcs can be formed between the distal ends of the electrodes. The tubular configuration of the gas supply means allows the protective gas to be supplied at least to the active regions of the electrodes. The adhesive bonding-in of electrodes is a simple and economical measure for fastening the electrodes and insulating them relative to one another.

In another embodiment the electrodes are arranged in the lumen, in each case embedded in an insulation layer, opposing one another and set apart from one another, in the direction of extension of the instrument. In this case, either the gas supply means forms—as described above—the insulation bed or the electrodes are explicitly sheathed and suspended in the lumen.

The first electrode is generally arranged in the lumen in the direction of extension of the instrument and the second electrode is arranged coaxially with the first electrode, set apart therefrom, in the lumen by at least one insulation layer being arranged in such a way that the electrodes are separated from one another outside their desired active regions. For this purpose, the first electrode can for example be surrounded by an insulation layer, or else the second, tubular electrode is embedded within the tubular gas supply means and thus insulated from the first electrode. Owing to the coaxial configuration of the electrodes (pin electrode, pipe electrode or annular electrode), branching-on of the arcs is possible, so that a larger front is available for the formation of heat.

Generally, the gas supply means comprises at least two lumens which are separated from one another, the electrodes being arranged in a respective lumen, and thus set apart from one another in the direction of extension of the instrument. An instrument embodied with two lumens allows the electrodes to be positioned in a simple manner; at the same time, different fluids, including for example a rinsing liquid, can be supplied, in addition to the protective gas, via the two (and if appropriate more than two) lumens.

The electrodes (in particular the active regions thereof) can be arranged parallel to one another. In another embodiment the electrodes are arranged in such a way that at least the distal ends of the electrodes are arranged diverging from one another (in principle curved apart from one another) so as to form elongated arcs which can be directed onto the tissue. As a result of this possibility of altering the electric field, the arc bulges forward in the direction of the tissue to be treated, so that the operator does not have to bring the instrument (in particular under endoscopic conditions) too close to the tissue.

Generally, the distal ends of the electrodes are arranged outside the lumen or lumens. In other words, the distal ends protrude from the gas supply means. The arcs are therefore formed in a free space between the electrodes and the tissue, so that the heat of the arcs can be transferred unimpeded toward the tissue.

Alternatively, it is possible for the distal ends of the electrodes to be arranged inside the lumen or lumens, so that the arcs can be formed at least partly inside the lumen or lumens. The gas supply means at the distal end of the instrument can be provided with outlet openings which are arranged in such a way that the heat generated by the arcs can be brought up to the tissue to be treated. In this case the electrodes would therefore not protrude from the instrument, but are arranged so as to be protected in the gas supply means. The instrument itself, or the distal end, then serves at the same time as a spacer, so that contacting of the electrodes with the tissue is not possible. This facilitates handling of the instrument, because in this case direct contact of electrodes and tissue to be treated can be avoided even in the event of any awkward handling of the instrument.

Specifically in the case of this embodiment it can be advantageous for the gas supply means to have at the distal end of the instrument outlet openings which are arranged in such a way that the heat generated by the arcs can be brought up to the tissue to be treated. That means that the instrument has lateral recesses to allow even better transfer of heat. For this purpose, the instrument can be constructed so as to be perforated in the distal region or have mutually set-apart webs or a similar lattice structure.

Another embodiment provides for the instrument to be constructed in such a way that a spacer can be arranged at the distal end, so that the instrument can be held at a predetermined spacing from the tissue to be treated. This on the one hand prevents direct contacting with the tissue (uncontrolled introduction of current, burning of tissue, burning of the electrodes onto the tissue) and on the other hand also provides (if appropriate) sufficient spacing between the arc and tissue. The size of the spacer is therefore arranged in such a way that it ensures the spacing, which is suitable for the transfer of heat, between the arc and tissue, wherein it can for example be connected in one piece with the instrument or can be attached thereto if required.

A particular challenge is presented by the insulation of the electrodes relative to one another and the resultant capacity of the arrangement and also the dielectric losses produced in the insulating material or the insulation layer. That applies in particular to probes for endoscopic applications. For this purpose, an output filter of the high-frequency generator is embodied in such a way that the arrangement of the electrodes relative to one another allows capacitive effects which occur to be compensated for. That is to say, in particular relatively high capacitances of relatively small and if appropriate coaxially constructed probes can be included in the filter and thus compensated for.

As indicated above, the bipolar surgical instrument can be constructed in such a way that it is suitable for endoscopic applications. For example, in the case of minimally invasive intervention, the instrument is embodied in such a way that at least the gas supply means can be brought up to the operating area for example through an instrument channel of an endoscope via a body opening. The endoscope inserted into the organ to be examined or into the body cavity is a flexible or rigid pipe having a plurality of channels. It is then possible to bring, in addition to the above-described (APC) probe, various working means, for example further surgical instruments, up to the operating area via the endoscope which usually has a plurality of lumens. In addition, it is also possible through the lumens to carry out rinsing, suction extraction or to take a tissue sample. In addition, the endoscope has an optical system in order to be able to monitor the treatment via imaging methods.

It is also possible to construct the instruments in such a way that they can be used for open surgery. In this case too, the instruments offer the advantage that patients are subjected to minimal stress owing to reduced or eliminated current introduction.

In yet another embodiment, a means for a magnetic blow-out, in particular a blow magnet, is arranged on the instrument so as to form elongated arcs which can be directed onto the tissue. That is to say, even relatively weak magnetic fields can cause migration of the arc to the electrodes at a speed corresponding to the current frequency (in a magnetic DC field) and bulging (in a synchronised AC field). The arrangement can be embodied in such a way that the magnetic field oscillates at the current alternating frequency in order to keep the Lorentz force constant (and thus to allow bulging in a direction toward the tissue). This can be achieved using an electromagnet. It is thus possible to advance arcs in the direction of the tissue, so that the tissue can be heated and without the instrument having to be brought up too close to the tissue.

In one embodiment the power source, i.e. the high-frequency generator, is embodied in such a way that it can be allocated to a control means for controlling the current required for forming the arcs, the control means being embodied in such a way that the current can be controlled or regulated for the purposes of the automatically controlled treatment sequence. This is carried out by means of an arc monitor and/or a current monitor which can be allocated to the control means, so that the current can be controlled or regulated as a function of a detected arc or as a function of a detected current value. The corresponding further course of the machining can be controlled or regulated based on the detection of arcs, so that an operator does not have to make decisions in this regard.

In terms of the method, the object is achieved in that, in a method for electrosurgical treatment of tissue with a bipolar surgical instrument having an electrode means, which is connected to a high-frequency generator for generating a high-frequency current, at a distal end of the instrument with at least a first electrode and a second electrode, and a pipe, a tubular probe or a similar gas supply means with a lumen, the following steps are provided:

bringing the instrument up to the tissue to be treated,

positioning the instrument in such a way that the tissue can be treated by means of the electrodes,

supplying argon or a similar inert gas at least into a space between the first electrode and the second electrode, by means of the gas supply means, so that between the first electrode and the second electrode arcs can be formed in a protective gas atmosphere,

forming electric arcs between the first and second electrode, so that the tissue can be heated in a currentless manner at least partly by heat generated by the arcs.

If the bipolar surgical instrument described above is used, this method allows tissue to be heated without difficulty and devitalized to a desired degree. If appropriate, allowance can be made—while the tissue is still moist—for a slight introduction of current, as arcs could then also ignite between the electrodes and the tissue. At the latest in an advanced stage of the treatment, once the tissue has already partly dried out, the current introduced is greatly reduced, if appropriate even completely eliminated. The tissue to be treated is then merely coagulated, via the heat generated by the arcs.

The extent to which the arcs are produced mainly between the electrodes depends on the spacings between the electrodes and between the electrodes and the tissue. As described above, spacers can in this case serve to adhere to these spacings in accordance with the desired treatment, without the operator having to handle the instrument in an excessively precise manner.

### DETAILED DESCRIPTION OF THE INVENTION

In the subsequent description the same reference numerals will be used for like and equivalent parts.

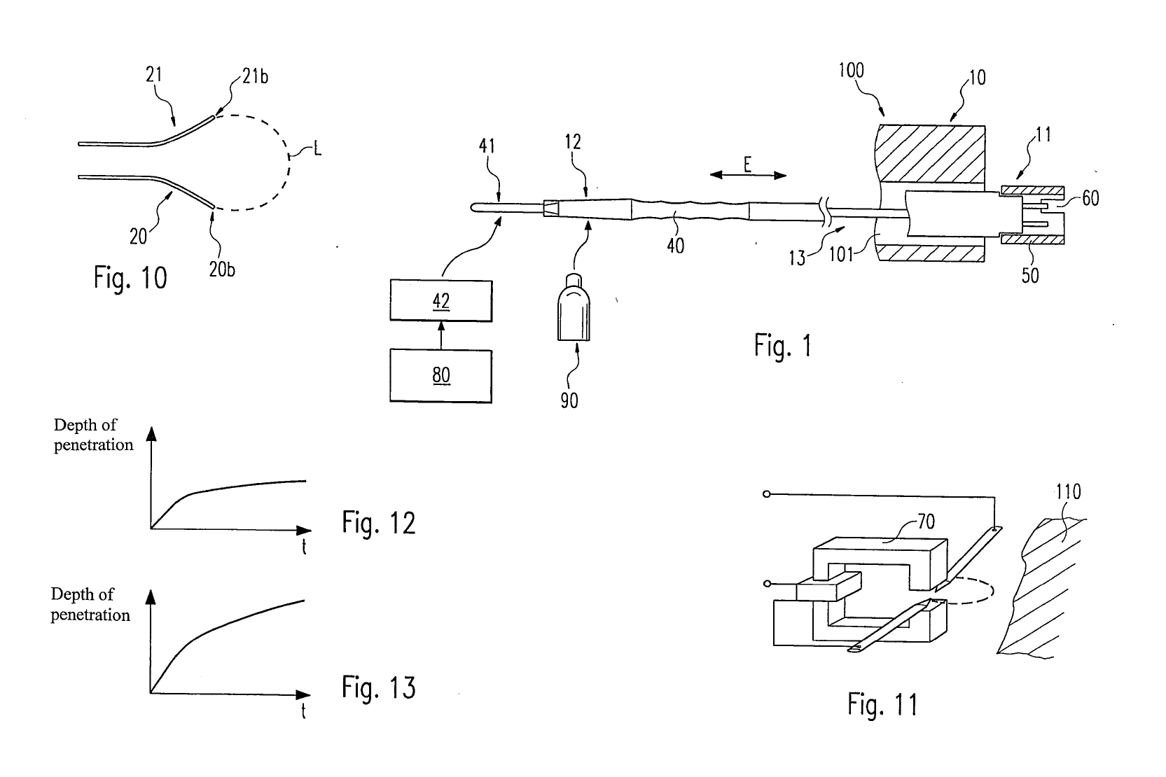

FIG. 1 shows an embodiment of a bipolar surgical instrument 10 with a current connection means 41 and a gripping means 40 at a proximal end 12 of the instrument 10, the instrument being connected to a power source 42 and a gas source 90.

The bipolar surgical instrument allows tissue 110 to be treated, in that between a first electrode 20 and a second electrode 21 of an electrode means arcs L are ignited and the tissue 110 is devitalized by means of the heat generated. For this purpose, the electrodes 20, 21 are arranged at a distal end 11 of the instrument 10 in such a way that arcs L ignite between desired active regions 20b, 21b of the electrodes 20, 21. In this embodiment the electrodes are arranged set apart from one another, but parallel to one another. The parallel arrangement allows the formation of an arcuate arc, so that the transfer of heat to the tissue is facilitated.

Generally, the instrument 10 is embodied with a gas supply means 13, so that the arcs L can ignite owing to a suppliable protective gas, for example argon, in a protective gas atmosphere. As the instrument is embodied, as shown in this exemplary embodiment, in a pipe-shaped or tubular manner, the pipe or the tube forms the gas supply means 13. Protective gas therefore surrounds the electrodes 20, 21 and the arcs L ignite in the safe protective gas atmosphere. This is for example necessary in order to keep explosive gases located in body cavities away from the ignition region. The gas, for example argon, originates from the gas source 90 to which the instrument 10 can be connected, if appropriate via a corresponding surgical means.

In this embodiment the instrument is introduced into a working channel 101 of an endoscope 100 and can therefore be brought up to the tissue 110 to be treated via a body opening. In the case of minimally invasive interventions there is thus no need to open up the patient's body. Nevertheless, it is also possible to use the instrument in open surgery.

As the FIG. 1 shows, current connection means 41 is provided on the gripping means 40 to improve handling of the instrument. The instrument 10 can be connected to the high-frequency generator 42 via the current connection means 41 for generating high-frequency current. The high-frequency generator 42 is embodied in such a way that it can be connected to a control means 80, so that for example the current can be controlled and thus the treatment sequence proceeds, if appropriate, automatically. The formation of arcs L can also be detected and further controlling of the treatment sequence (current control, voltage control) allowed.

The bipolar surgical instrument 10 has at its distal end 11 the electrode means, distal ends 20a, 21a of the electrodes 20, 21 protruding in each case from the pipe or the tube and extending in the direction of extension E of the instrument 10, i.e., in the axial direction. Thus, the ends of the electrodes are also arranged parallel to one another, so that the arcs L can ignite (the ignition is carried out at the active regions of the electrodes). The heat thus generated is then utilized, for example, to coagulate the tissue. It is in this way possible to reach much lower depths of devitalization (depths of penetration) into the tissue than would be possible using conventional instruments and the targeted introduction of current. FIG. 12 shows the (exemplary) depth of coagulation into the tissue over time t, such as may be expected using the instruments (which focus on the utilisation of heat). FIG. 13 shows the penetration characteristic in instruments according to the prior art, current being introduced into the tissue in a targeted manner (in this case too, the depth of devitalization or depth of penetration is shown over time t). It is thus clear that the bipolar surgical instrument 10 allows the process to be carried out in a much gentler manner, causing less damage to tissue, but not sacrificing efficiency. The course of coagulation can be estimated much more accurately and the development of heat in the tissue can be metered more effectively.

As a formation of arcs between the electrodes 20, 21 and the tissue 110 is to be avoided, a defined spacing of the instrument 10 from the tissue is required. This can be done in a simple manner by means of a spacer 50. The spacer 50 can for example be attached to the distal end 11 of the instrument 10 (e.g. to the gas supply means), so that the operator no longer has to handle the instrument in an excessively precise manner.

The spacer 50 can be connected in one piece with the instrument 10 or else be provided as an explicit component. If appropriate, the spacer can have outlet openings 60 or similar perforations or recesses, so that the heat can be transferred therethrough.

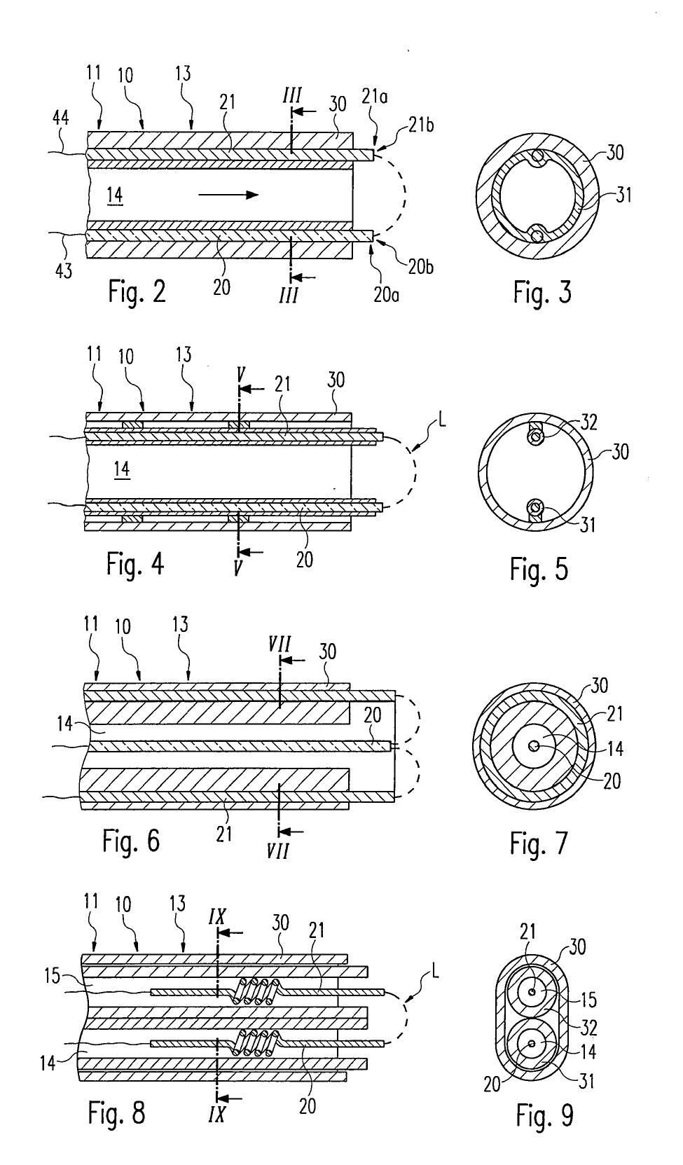

FIG. 2 shows the distal end 11 of the bipolar surgical instrument 10 shown in FIG. 1. The electrode arrangement is shown in this case in greater detail. The first and the second electrode 20, 21 diametrically oppose one another on an inner lateral surface of the pipe or tube of the instrument. In order to eliminate interaction of the electrodes 20, 21 inside the instrument 10, the electrodes are embedded into an insulation layer 30. This electrically and thermally insulating layer may for example be an adhesive layer by means of which the electrodes are adhesively bonded to the inner surface of the pipe or tube.

In order to form the arcs L, the active regions 20b, 21b of the electrodes 20, 21 protrude from the pipe without insulation.

According to FIG. 2 the electrodes 20, 21 are arranged relative to one another in such a way that between them a lumen 14 is embodied for supplying gas. Via this lumen the gas, for example argon, can be supplied and sweeps around the electrodes. The protective gas surrounds, in particular, the distal ends 20a, 21a of the electrodes 20, 21, so that—as discussed above—the arcs ignite in the protective gas atmosphere. The arrow illustrated in the lumen indicates the direction of the supply of fluid.

FIG. 3 is a sectional view of the distal end 11 of the bipolar surgical instrument 10 along the sectional line III-III from FIG. 2. The adhesive layer 31 (or else any other type of insulation) completely covers in this case the inner surface of the distal pipe end, so that, apart from their active regions, the electrodes are embedded in the layer.

It is also possible to provide recesses in the pipe or the probe, i.e., the gas supply means, so that the electrodes can be inserted into the insulating material of the gas supply means and also extend therefrom in the longitudinal direction E of the instrument.

FIGS. 4 and 5 show a similar embodiment to that shown in FIGS. 2 and 3. These figures also show in each case the distal end 11 of the instrument 10, the two electrodes 20, 21 each being sheathed by an insulation layer 31, 32 and being arranged in the lumen 14 in the direction of extension E of the instrument 10. The sheathed electrodes can be fastened to the interior of the pipe, for example via holding elements, and thus be positioned, for example, diametrically opposing one another. This is particularly clear in FIG. 5, which shows a section along the line V-V from FIG. 4. In this case too, the active regions 20b, 21b of the electrodes protrude, again, from the instrument in order to form the arcs. The pipe, i.e. the gas supply means, itself forms an insulation layer 30.

The embodiments according to FIGS. 1-5 allow the arcs to be formed in a clearly defined manner between the ends of the electrodes.

FIG. 6 shows a further embodiment of a bipolar surgical instrument 10, the distal end 11 of the instrument 10 being shown in section; FIG. 7 shows the distal end of the instrument according to FIG. 6 in section along the line VII-VII from FIG. 6. A coaxial arrangement of the electrodes 20, 21 is provided in this case, i.e. the first electrode 20 is arranged substantially centrally in the pipe 13 of the instrument 10, i.e. the gas supply means, while the second, tubular electrode 21 is arranged coaxially with the first, providing a spacing. Owing to the spacing, the lumen 14, which is required for supplying gas, is embodied between the two electrodes 20, 21. The second electrode 21 is embedded in the probe made of insulating material 30, so that outside the distal ends 20a, 21a of the electrodes 20, 21 no interaction can take place between them.

FIG. 8 shows a further embodiment of a bipolar surgical instrument 10, the distal end 11 of the instrument being illustrated in section; FIG. 9 shows the distal end of the instrument according to FIG. 8 in section along the line IX-IX from FIG. 8. This arrangement shows a probe having an oval cross section (see FIG. 9), two lumens 14, 15 being provided in the probe made of insulating material 30. The lumens are each surrounded by insulation layers 31, 32, so that the electrodes 20, 21 guided in the respective lumens 14, 15 are insulated from one another. In order to secure the electrodes in the lumens, the lumens have a helical region to allow clamping of the electrodes inside the lumens, in each case on the insulating sheathing 31, 32. Thus, the electrodes are securely fixed in the instrument.

A plurality of lumens 14, 15 allow, for example, various fluids, e.g., a rinsing liquid, to be brought up to the operating area. In particular, the electrodes do not have to be explicitly set apart from one another and insulated, as this is provided by the two lumens anyway.

Moreover, the first electrode 20, which is provided in the coaxial arrangement, (FIGS. 6 and 7) can also be fixed in the instrument by means of the helical region.

The electrodes are connected in all the exemplary embodiments to current supply means such as feed lines (or discharge lines) 43, 44, so that they can be connected to the high-frequency generator.

FIG. 10 is a schematic illustration of the ends 20a, 21a of the electrodes, said ends being arranged diverging from one another in their active regions 20b, 21b. It is thus possible to form an arc L, which is elongated relative to the above-described arrangements (distal ends, arranged parallel to one another, of the electrodes), in the direction of the tissue 110, so that the transfer of heat to the tissue is simplified.

FIG. 11 shows in simplified form a further possibility for forming arcs which are elongated in the direction of the tissue. For this purpose, a magnet 70 is arranged on the instrument 10 or at the distal end 11 thereof in such a way that the Lorentz force causes bulging of the arc L toward the tissue 110 to be treated. An electromagnet ensures that the Lorentz force allows the arcs to bulge in the desired direction even in the case of an alternating current. The means 70 for magnetic blow-out therefore allows the defined formation of arcs.

As the foregoing discussion reveals, an electrosurgical arrangement therefore allows tissue to be treated while greatly reducing an introduction of current, only the heat generated by the bipolar surgical instruments being utilised. This spares the tissue to a high degree and unnecessary burning and devitalisation phenomena are avoidable. If appropriate, allowance should be made for a slight introduction of current into the tissue at the start of a treatment, while the tissue is still moist; however, at the latest in the further course of the treatment, further devitalisation is allowed, primarily owing to the heat formed by the arcs.

Moreover, it should be noted that the hatching shown in the figures is not intended to indicate the nature of the material. Thus, for example, one electrode (although generally made of the same material as the other electrode) is illustrated with hatching made up of a broken and solid line, while the other electrode is hatched merely by means of solid lines. This is intended to allow the first and second electrode to be differentiated. The insulation layers which are necessary for forming the instruments can for example be made of a plastics material or of ceramic. In this case, the insulation layers are primarily made of electrically insulating and generally also of thermally insulating material.

It is noted that the above description and drawings are exemplary and illustrate preferred embodiments that achieve the objects, features and advantages of the present invention. It is not intended that the present invention be limited to the illustrated embodiments. Any modification of the present invention which comes within the spirit and scope of the following claims should be considered part of the present invention.

1. A bipolar surgical instrument for electrosurgical treatment of tissue comprising:
electrode means connected to a high-frequency generator for generating a high-frequency current at a distal end of the bipolar surgical instrument with at least a first electrode and a second electrode for forming electric arcs between the first and second electrodes; and
gas supply means with at least one lumen for supplying argon or a similar inert gas at least into a space between the first electrode and the second electrode, so that the arcs can be formed in a protective gas atmosphere comprising argon or similar inert gas supplied by the gas supply means,
wherein the first and second electrodes are arranged relative to one another in such a way that the tissue can be heated in a currentless manner at least partly by heat generated by the arcs.

2. The bipolar surgical instrument of claim 1, wherein the first and second electrodes are positioned at the distal end of the instrument in such a way that they are separated from one another by the at least one lumen or at least one insulation layer, at least distal ends of the electrodes each forming an active region where the arcs can be formed between the first and second electrodes.

3. The bipolar instrument of claim 1, wherein the first and second electrodes are arranged in the lumen opposing one another in the direction of extension of the bipolar surgical instrument and separated from one another by the lumen of the gas supply means and at least one insulation layer.

4. The bipolar surgical instrument of claim 1, wherein the electrodes are arranged in the lumen and each embedded in an insulation layer, opposing one another and set apart from one another, in the direction of extension of the bipolar surgical instrument.

5. The bipolar surgical instrument of claim 1, wherein the first electrode is arranged in the lumen in the direction of extension of the instrument and the second electrode is arranged coaxially with the first electrode, the first and second electrodes being separated in the lumen by at least one insulation layer.

6. The bipolar surgical instrument of claim 1, wherein the gas supply means comprises at least two lumens separated from one another in which the first and second electrodes are arranged, respectively, so that they are set apart from one another in the direction of extension of the bipolar surgical instrument.

7. The bipolar surgical instrument of claim 1, wherein the electrodes are arranged in such a way that at least distal ends of the electrodes diverge from one another so as to form elongated arcs which can be directed onto the tissue.

8. A bipolar surgical instrument of claim 6, wherein distal ends of the electrodes are arranged outside the lumens.

9. The bipolar surgical instrument of claim 6, wherein distal ends of the electrodes are arranged inside the lumens, so that the arcs can be formed at least partly inside the lumens.

10. The bipolar surgical instrument of claim 1, wherein the gas supply means has at the distal end of the bipolar surgical instrument outlet openings which are arranged in such a way that the heat generated by the arcs can be directed to the tissue to be treated.

11. The bipolar surgical instrument of claim 1, further comprising a spacer arranged at the distal end of the bipolar surgical instrument, so that the bipolar surgical instrument can be held at a predetermined spacing from the tissue to be treated.

12. The bipolar surgical instrument of claim 1, further comprising an output filter of the high-frequency generator operable to compensate for capacitive effects occurring as a result of the arrangement of the first and second electrodes.

13. The bipolar surgical instrument of claim 1, wherein the bipolar surgical instrument is adapted for use in open surgery.

14. The bipolar surgical instrument of claim 1, wherein at least the gas supply means can be brought up to the tissue to be treated through an instrument channel of a rigid or flexible endoscope.

15. The bipolar surgical instrument of claim 1, further comprising means for a magnetic blow-out so as to form elongated arcs which can be directed onto the tissue.

16. The bipolar surgical instrument of claim 1, further comprising control means for for controlling the current generated by the high-frequency generator required for forming the arcs such that current can be controlled or regulated for the purposes of an automatically controlled treatment sequence.

17. A method for electrosurgical treatment of tissue using a bipolar surgical instrument comprising first and second electrodes located at a distal end of the bipolar surgical instrument and connected to a high-frequency generator for generating a high-frequency current, and a gas supply means with at least one lumen, the method comprising the steps of:
bringing the bipolar surgical instrument up to the tissue to be treated,
positioning the bipolar surgical instrument in such a way that the tissue can be treated by means of the electrodes,
supplying argon or a similar inert gas at least into a space between the first electrode and the second electrode, by means of the gas supply means, so that between the first electrode and the second electrode arcs can be formed in a protective gas atmosphere of the argon or similar inert gas, and
forming electric arcs between the first and second electrodes, so that the tissue can be heated in a currentless manner at least partly by heat generated by the arcs.

18. The method of claim 17, wherein the bipolar surgical instrument further comprises a spacer arranged at the distal end of the bipolar surgical instrument and the positioning step further comprises using the spacer to position the first and second electrodes at a predetermined spacing from the tissue.

19. The bipolar surgical instrument of claim 1, wherein the first and second electrodes are set apart from each other along an inner surface of said lumen and electrically isolated from each other along an entire length of the lumen by an insulating layer covering the inner surface of the lumen and the first and second electrodes.

20. A bipolar surgical instrument for electrosurgical treatment of tissue comprising:
a probe for supplying argon or a similar inert gas to tissue to be treated comprising tubular insulating material;
a first electrode arranged substantially centrally in the interior of the probe;
a second, tubular electrode arranged coaxially with the first electrode and embedded in the tubular insulating material such that no portion of the second electrode is exposed to the interior of the probe, wherein the first and second electrodes are arranged relative to one another in such a way that the tissue to be treated can be heated in a currentless manner at least partly by heat generated by electric arcs formed between the first and second electrodes in a protective gas atmosphere comprising argon or similar inert gas supplied by the probe.
